(19) United States
(12) Patent Application Publication
(10) Pub. No.: US 2019/0175283 A1
(43) Pub. Date: Jun. 13, 2019

(54) PINLESS FEMORAL TRACKING

(71) Applicant: THINK SURGICAL, INC., Fremont, CA (US)

(72) Inventors: Daniel P. BONNY, Fremont, CA (US); Joel ZUHARS, Fremont, CA (US)

(21) Appl. No.: 16/322,982

(22) PCT Filed: Aug. 10, 2017
(86) PCT No.: PCT/US2017/046250

§ 371 Date: Feb. 4, 2019

Related U.S. Application Data
(60) Provisional application No. 62/373,066, filed on Aug. 10, 2016.

(51) Int. Cl. A61B 34/20; A61B 90/00; A61B 34/10

(52) U.S. Cl. CPC A61B 34/20; A61B 90/39; A61B 34/10; A61B 2090/3945; A61B 2034/2068; A61B 2034/105; A61B 2034/104; A61B 2034/2055

(57) ABSTRACT

A system and method are provided to optically track a subject's anatomy without the need for the use of invasive pins and screws currently used to support and attach six-degree-of-freedom fiducial arrays directly to the bone. The system and method is especially advantageous for surgical procedures involving brittle or osteoporotic bone or any other procedure where the preservation of surrounding healthy bone is desirable. The system and method are advantageous for computer-assisted total knee arthroplasty, revision knee arthroplasty, and other medical procedures where the position and orientation (POSE) of bone surface cuts planes are critical for successful placement and correct alignments of joint implants. Often, these procedures require a form of external support to reduce the motion of the bones relative to the computer-assisted device.

### CROSS-REFERENCE TO RELATED APPLICATION

This application claims priority of U.S. Provisional Patent Application Ser. No. 62/373,066 filed Aug. 10, 2016, which is incorporated herein by reference.

### TECHNICAL FIELD

The present invention generally relates to computer assisted surgery, and more specifically to a system and method for pinless tracking during orthopedic surgical procedures.

### BACKGROUND

Total knee arthroplasty (TKA) is a surgical procedure in which the articulating surfaces of the knee joint are replaced with prosthetic components, or implants. TKA requires the removal of worn or damaged articular cartilage and bone on the distal femur and proximal tibia. The removed cartilage and bone is then replaced with synthetic implants, typically formed of metal or plastic, to create new joint surfaces.

The position and orientation (POSE) of the removed bone, referred to as bone cuts or resected bone, determines the final placement of the implants within the joint. Generally, surgeons plan and create the bone cuts so the final placement of the implants restores the mechanical axis or kinematics of the patient's leg while preserving the balance of the surrounding knee ligaments. Even small implant alignment errors outside of clinically acceptable ranges correlate to significantly worse outcomes that are associated with increased rates of revision surgery. In TKA, creating the bone cuts to correctly align the implants is especially difficult because the femur requires at least five planar bone cuts to receive the femoral prosthesis. The planar cuts must be aligned in at least five degrees of freedom to ensure a proper orientation: anterior-posterior translation, proximal-distal translation, external-internal rotation, varus-valgus rotation, and flexion-extension rotation. Mal-alignment in any one of these planar cuts or orientations may have drastic consequences on the final result of the procedure and the wear pattern of the implant, resulting in reduced functionality and decreased implant longevity. To obtain accurate and durable implantation, one must not only achieve correct alignment of the prosthesis with the bone, but also correct positioning of the prosthesis within the bone to achieve reliable and durable anchoring.

In the medical field, tracking systems have been utilized with medical devices to assist surgeons in performing precision surgery. Typical configurations and methods for tracking objects are well known in the art. One such method exploits the emission or reflection of signals (light, radiofrequency, infrared) attached to an object that are detected by receivers (photodiodes, CMOS or CCD cameras). The signals are detected by receivers and then processed to locate the position and orientation (POSE) of the object. Alternatively, or in combination with signal detection, receivers may detect patterns, sequences, shapes, or characters attached to an object that may also be processed to determine the POSE of the object. In particular, optical tracking systems utilizing infrared light are commonly used due to their accuracy and adaptability.

During computer assisted surgery, fiducial marker arrays may be used to track rigid objects, including the operative anatomy, such as the femur and tibia. In order to track a rigid body using an optical tracking system in complex surgical procedures including total knee arthroplasty, a rigid array of fiducial markers sufficient to resolve six degrees of freedom may be required. A marker array that is sufficiently accurate for computer assisted surgery or robotic-assisted surgery can be quite large, requiring invasive pins or screws drilled into the bone to ensure that there is minimal deflection between the bone and the marker. The securement of pins and screws is time-consuming and invasive. Additionally, such pins and screws, which are typically greater than 3.2 mm in diameter, may induce tissue trauma that can result in bone fracture.

Thus, there is a need for a system and method to attach fiducial arrays that maintain a minimal deflection between a subject's bone and marker while avoiding the use of invasive pins and screws.

### SUMMARY OF THE INVENTION

A surgical system is provided for tracking a bone of a subject during a surgical procedure. The surgical system includes a brace with a securement adapted for holding a portion of a limb of the subject, and a fiducial array with a series of fiducial markers attached to the brace. The system further includes one or more single-LED (light emitting diode) markers each individually suspended by an extension, where the extension is inserted into a portion of a bone of the subject, a tracking system adapted to track the position and orientation (POSE) of the fiducial array and the one or more single-LED markers; and a computing system, interfaced with the tracking system, programmed to: store at least one of a position and an orientation of one or more reference features with respect to the fiducial marker array on the brace; and compensate for the remaining degrees of freedom between the brace and the one or more reference features based on the tracked spatial relationship between the fiducial marker array on the brace and the one or more single-LED markers inserted on the bone.

A method is provided for tracking a bone of a subject that includes placing a portion of a limb of the subject in a brace, and securing the portion of the limb to the brace with a securement to establish a position and orientation of a fiducial marker array attached to the brace. The method further includes inserting one or more single-LED (light emitting diode) markers with extensions into the subject's bone, and activating a tracking system to track the position and orientation (POSE) of the marker array and the one or more single-LED. At least one position and orientation is stored of one or more reference features with respect to the marker array on the brace for use as a base reference frame, and tracking the bone, where the one or more single-LED markers are used to compensate for the remaining degrees of freedom between the brace and one or more reference features.

A surgical system is provided for monitoring motion of a bone of a subject during a surgical procedure. The system includes a brace with a securement adapted for holding a portion of a limb of the subject, and a fiducial array with a series of fiducial markers fixedly attached to the brace. The system further includes one or more single-LED (light emitting diode) markers each individually suspended by an extension, where the extension is inserted into a portion of the bone, and a tracking system adapted to track the position and orientation (POSE) of the fiducial array and the one or more single-LED markers. A computing system is interfaced with the tracking system, and is programmed to: store one or more reference features with respect to the fiducial marker array on the brace, and notify a user if the tracking system detects motion between the one or more single-LED markers and the fiducial marker array.

### BRIEF DESCRIPTION OF THE DRAWINGS

The present invention is further detailed with respect to the following drawings that are intended to show certain aspects of the present of invention, but should not be construed as limit on the practice of the invention, wherein:

FIG. 1 illustrates a subject's leg positioned and secured in a foot holder with a securement having a fixed fiducial array attached, and a series of individual fiducial markers inserted or affixed to the subject's femoral bone in accordance with embodiments of the invention;

FIG. 2 illustrates a surgical system in the context of an operating room (OR) with the subject positioned on the operating table as shown in FIG. 1 in accordance with embodiments of the invention; and

FIG. 3 illustrates a virtual pin plane defined relative to a planned cut plane on a three-dimensional model of a bone in accordance with embodiments of the invention.

### DETAILED DESCRIPTION

The present invention has utility as a system and method to optically track a subject's anatomy during a procedure without the need for the use of invasive pins and screws currently used to support and attach six-degree-of-freedom fiducial arrays directly to the bone. The system and method is especially advantageous for surgical procedures involving brittle or osteoporotic bone or any other procedure where the preservation of surrounding healthy bone is desirable. In particular, the system and method are advantageous for computer-assisted total knee arthroplasty and revision knee arthroplasty where the position and orientation (POSE) of bone surface cuts planes are critical for successful placement and correct alignments of joint implants. Often, these procedures require a form of external support to reduce the motion of the bones relative to the computer-assisted device. However, it should be appreciated that other medical applications may exploit the subject matter disclosed herein.

Embodiments of the present invention provide the ability to track a rigid body, such as a bone, using an optical tracking system, where a rigid array of fiducial markers sufficient to resolve six degrees of freedom (i.e., a six-degree-of-freedom fiducial array) is sufficiently accurate for computer assisted surgery or robotic-assisted surgery without requiring invasive pins or screws for attaching the arrays directly to a bone.

As used herein the terms “rigid” or “rigidly” are defined to mean an object that does not deviate in position relative to a base structure, the base structure specifically being a bone or a brace to which it is attached through the range of motions the base structure experiences during a surgical procedure.

The following description of various embodiments of the invention is not intended to limit the invention to these specific embodiments, but rather to enable any person skilled in the art to make and use this invention through exemplary aspects thereof. As used herein, a patient or subject is defined as a human, a non-human primate; or an animal of a horse, a cow, a sheep, a goat, a cat, a rodent and a bird; or a cadaver of any of the aforementioned.

It is to be understood that in instances where a range of values are provided that the range is intended to encompass not only the end point values of the range but also intermediate values of the range as explicitly being included within the range and varying by the last significant figure of the range. By way of example, a recited range from 1 to 4 is intended to include 1-2, 1-3, 2-4, 3-4, and 1-4.

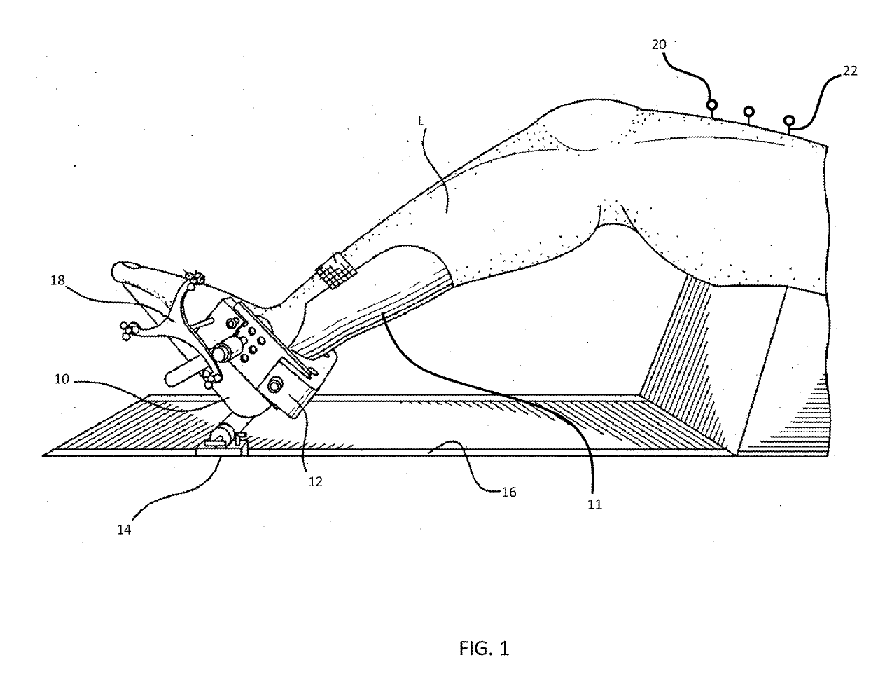

Referring now to the figures, FIG. 1 illustrates a leg, L of a subject positioned and secured in a foot holder 10 of a knee brace 11 with a securement 12 having a fixed fiducial marker array 18 attached to the brace 11, and a series of individual fiducial markers 20 inserted or affixed to the femoral bone of the subject. The foot holder 10 is movable secured and locked into place with an attachment bracket 14 to a positioning rail 16 that is laid on the surface of an operating table below the patient. An example of the knee brace 11 as shown in FIG. 1 is a DeMayo knee brace as disclosed in U.S. Pat. No. 7,380,299 issued Jun. 3, 2008 to DeMayo. Other examples of external supports or limb positioning systems like the DeMayo knee brace for use with the present invention include the Soloarc leg positioning system (Match Grade Medical LLC, Neenah, Wis.), the Trimano® Arm Holder (Arthrex, Inc., Naples, Fla.), the Hana® table (Mizuho OSI, Union City, Calif.), and similar limb positioning devices.

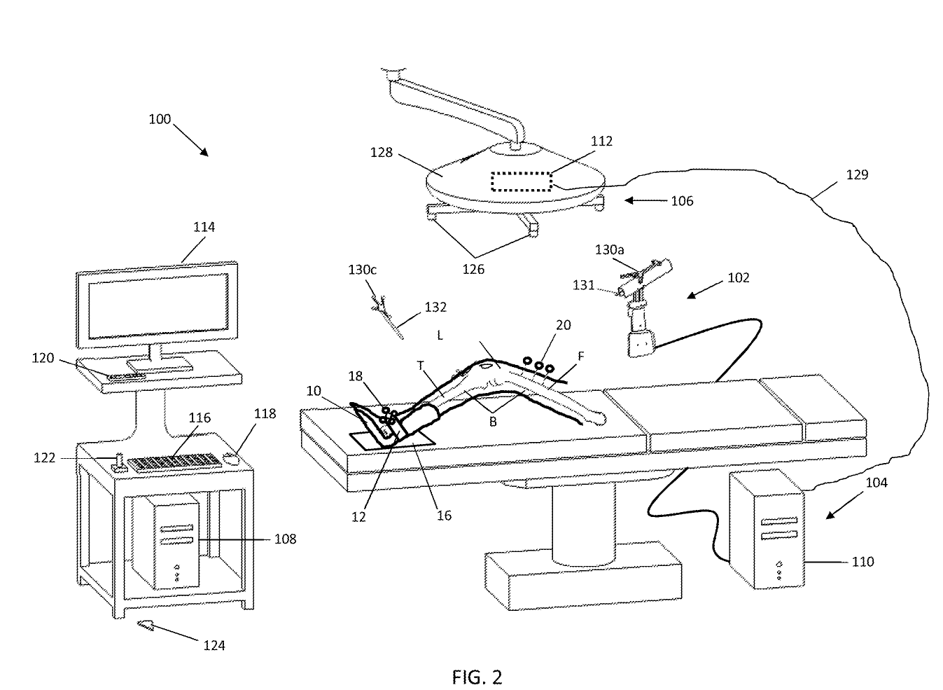

The marker array 18 is rigidly fixed with respect to the knee brace 11, and as such, the marker array 18 can be as large as necessary while still allowing rigid fixation to the brace 11. This alleviates the stress a large tracking array would otherwise impose on the patient's leg L in a traditional procedure. One or more single-light emitting diode (LED) markers 20 suspended by extensions 22, which may illustratively include tacks or small screws that in some inventive embodiments are inserted into the femur of the subject such that the single-LED markers 20 are visible to a tracking system (shown in FIG. 2 at reference numeral 106). In a particular embodiment, the extensions 22 have a diameter less than 3 mm and can be fully inserted into the bone of the patient, analogous to a thumbtack inserted into a tack board; however, here the thumbtack head is the single-LED marker. Each extension 22, is only supporting a single-LED marker 20, thus, there is minimal loading between the extension 22 and the femur. At least one of a position and an orientation of one or more reference features (i.e., a feature used as a reference for tracking an anatomical region of interest) is stored with respect to the marker array 18 on the knee brace 11 as the base reference frame. In a particular embodiment, the reference feature is one or more points collected on the bone, where the one or more points are stored in the marker array 18 reference frame. In another embodiment, the reference feature is a virtual bone model registered to the bone, where the POSE of the virtual bone model coordinate system is stored with respect to the marker array 18 reference frame. In another embodiment, the reference feature is a virtual implant model positioned with respect to at least one of a virtual bone model, one or more points collected on the bone, or a statistically shaped bone morphed model. The POSE of that virtual implant model coordinate system is stored with respect to the marker array 18 reference frame.

The one or more single-LED markers 20 on the femur are then used to compensate for the remaining degrees of freedom between the brace 11 and the femur. During a procedure, the tracking system 106 dynamically measures the base reference frame on the knee brace 11 and compensates for the remaining degrees of freedom by measuring the relationship between the knee brace 11 and the one or more single-LED markers 20. For example, it may be known the remaining degree of freedom between the bone and the knee brace 11 is purely translational along a certain axis. If the bone translates along that axis, then the translational component of the coordinates/transformation between the fiducial marker array 18 and the one or more reference features is updated based on the measured displacement between the fiducial marker array 18 and the single-LED marker 20 inserted on the bone. The remaining degrees of freedom between the bone and the knee brace may be determined or known from experimental data, simulations, or an expected kinematic relationship between the brace 11 and the bone (e.g., the tibia is expected to rotate about its longitudinal axis when secured in the brace 11).

The number of one or more single-LED markers 20 required depends on the degrees of freedom of motion between the brace 11 affixed to the patient's lower leg L and the patient's femur. If the motion is purely translational, then a single-LED marker 20 would be required (a point defines three degrees of freedom). If there is a single rotational degree of freedom without translation, then at least two of the single-LED markers 20 are required where the two single-LED markers are not placed on the rotational axis of the rotational degree of freedom. If there is a known axis of rotation between the brace and the bone, then a single-LED marker may compensate for the rotation because the single-LED marker will rotate with a constant radius about the axis of rotation. More single-LED markers 20 may be used to ensure redundancy. If three or more single-LED markers 20 are used then the femoral bone may be considered an uncalibrated fiducial marker array; however, the arrangement of the three or more single-LED markers 20 will be smaller and thus less accurate than the larger fixed marker array 18 on the brace 11. In a particular embodiment, the uncalibrated fiducial marker array may be calibrated with the tracking system by measuring and storing the relative positions or geometry of the three or more single-LED markers inserted on the bone prior to surgery.

### Computing System and Tracking System

The aforementioned method for tracking a bone is particularly useful with computer-assisted surgical devices. Examples of a computer-assisted surgical system include a 1-6 degree of freedom hand-held surgical system, an autonomous serial-chain manipulator system, a haptic serial-chain manipulator system, a parallel robotic system, a master-slave robotic system, or a navigated surgical device, as described in U.S. Pat. Nos. 5,086,401, 7,206,626, 8,560,047, 8,876,830, 8,961,536, 9,421,019 and U.S. Pat. App. No. 2013/0060278.

In a particular embodiment, with reference to FIG. 2, a surgical device 102 is controlled by commands from a computing system 104 to aid in the execution of a surgical plan. The computing system 104 may include a planning computer 108 including a processor; a device computer 110 including a processor; a tracking computer 112 including a processor; and peripheral devices. Processors operate in the computing system 104 to perform computations associated with the inventive system and method. It is appreciated that processor functions are shared between computers, a remote server, a cloud computing facility, or combinations thereof.

In a particular embodiment, the device computer 110 may include one or more processors, controllers, and any additional data storage medium such as RAM, ROM or other memory to perform functions related to the operation of the surgical device 102. For example, the device computer 110 may include software, data, and utilities to control the surgical device 102, receive and process tracking data, execute registration algorithms, execute calibration routines, provide workflow instructions to the user throughout a surgical procedure, as well as any other suitable software, data or utilities required to successfully perform the procedure in accordance with embodiments of the invention.

The planning computer 108, device computer 110, and tracking computer 112 may be separate entities as shown, or it is contemplated that their operations may be executed on just one or two computers depending on the configuration of the surgical system 100. For example, the tracking computer 112 may have the operational data to control the surgical device 102 without the need for a device computer 110. Or, the device computer 110 may include operational data to plan the surgical procedure without the need for the planning computer 108. In any case, the peripheral devices allow a user to interface with the surgical system 100 and may include: one or more user-interfaces, such as a display or monitor 114; and user-input mechanisms, such as a keyboard 116, mouse 118, pendent 120, joystick 122, foot pedal 124, or the monitor 114 may have touchscreen capabilities.

The planning computer 108 contains hardware (e.g., processors, controllers, and memory), software, data and utilities that are dedicated to aid a user in planning a surgical procedure, either pre-operatively or intra-operatively. This may include reading medical imaging data, segmenting imaging data, constructing and manipulating three-dimensional (3D) virtual models, generating virtual bone models using bone morphing techniques or digitization, storing and providing computer-aided design (CAD) files, planning the POSE of implants relative to the bone, defining virtual pin planes, and generating the surgical plan data for use with the system 100. The final surgical plan data may include an image data set of the bone, virtual bone models, bone registration data points, subject identification information, the desired POSE of the implants relative to the bone B, the POSE of one or more virtual planes defined relative to the bone B, and any tissue modification instructions such as a cut file or a bounding volume to create a desired modification to the bone. The final surgical plan is readily transferred to the device computer 110 and/or tracking computer 112 through a wired or wirelessly connection in the operating room (OR); or transferred via a non-transient data storage medium (e.g., a compact disc (CD), a portable universal serial bus (USB) drive) if the planning computer 108 is located outside the OR.

The tracking system 106 includes two or more optical receivers 126 to detect the position of fiducial markers 20. A set of fiducial markers 20 uniquely arranged on a rigid body is referred to herein as a fiducial marker array (18, 130a, 130b, 130c). Illustrative examples of the fiducial markers for the optical tracking system 106 may include: an active transmitter, such as an LED or other radiant energy emitter; a passive reflector, such as a plastic sphere with a retro-reflective film; or a distinct pattern or sequence of shapes, lines or other characters. An example of an optical tracking system is described in U.S. Pat. No. 6,061,644. The tracking system 106 may be built into a surgical light 128, located on a boom, a stand, or built into the walls or ceilings of the OR. The tracking system computer 112 may include tracking hardware, software, data and utilities to determine the POSE of objects (e.g., bones B, the surgical device 102) in a local or global coordinate frame. The POSE of the objects is also referred to herein as POSE data. An interface 129 is provided between the tracking system 106 and the computing system 104 to exchange tracking data therebetween. The interface 129 is appreciated to be wired or wireless using conventional protocols such as Wi-Fi. In a particular embodiment, the device computer 110 may determine the POSE data using the raw position data of the fiducial markers 20 detected directly from the optical receivers 126.

POSE data from the tracking system 106 is used by the computing system 104 to perform various functions. For example, the POSE of a digitizer probe 132 with an attached probe fiducial marker array 130c may be calibrated such that the probe tip is continuously known as described in U.S. Pat. No. 7,043,961. The POSE of the tip or axis of a tool pin 131 of the surgical device 102 may be known with respect to a device fiducial marker array 130a using a calibration method as described in U.S. Prov. Pat. App. 62/128,857. Registration algorithms are readily executed to determine the POSE and coordinate transforms between a bone B and the surgical plan, using the registration methods described in U.S. Pat. Nos. 6,033,415, and 8,287,522. For example, in a registration method, points on a patient bone may be collected from a tracked digitizer probe to transform the coordinates of a surgical plan to the coordinates of the bone.

The POSE data is used by the computing system 104 during the procedure to update the coordinate transforms between the surgical device 102, and the surgical plan registered to the bone B as the surgical device and bone B move in the workspace. The position of the bone B and the corresponding position of the surgical plan is updated as previously describe with respect to the fiducial marker array 18 on the knee brace 11 using the one or more single-LED markers. Therefore, a relationship between the POSE of the device 102, and the POSE of any coordinates defined in the surgical plan and registered to the bone B, is known by the computing system 104. In turn, the computing system 104, in some inventive embodiments supplies commands in real-time to the surgical device 102 to accurately execute the surgical plan. In the example shown, the bone(s) B are the tibia T and femoral F of the leg L of the subject. As was shown in FIG. 1 the leg L is held in place during a surgical procedure with the foot holder 10 and securement 12, which is locked into place via the attachment bracket 14 and rail 16. A fixed fiducial array 18 is rigidly attached to the securement 12 (or another portion of the brace 11). The one or more single-LED markers 20 are shown illustratively attached to the femoral bone F.

### Surgical Planning and Execution for a Total Knee Arthroplasty (TKA) Application

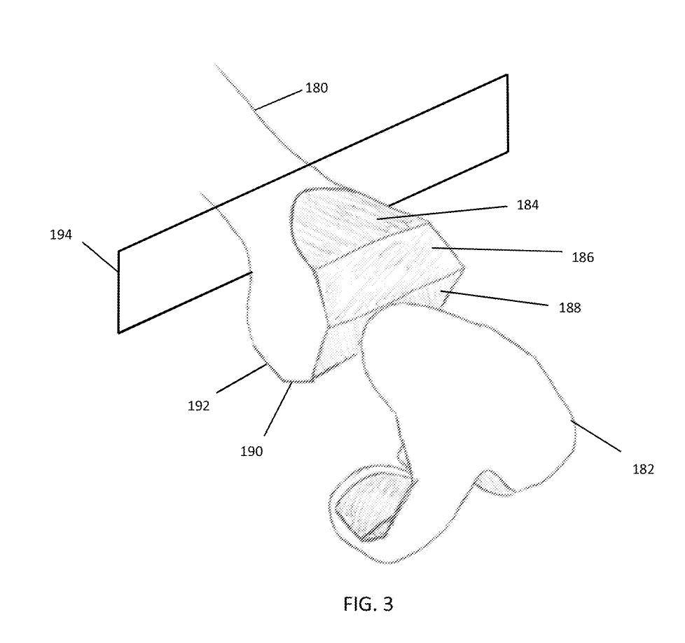

A surgical plan is created, either pre-operatively or intra-operatively, by a user using planning software. The planning software may be used to a generate three-dimensional (3-D) models of the subject's bony anatomy (i.e., virtual bone models) from a computed tomography (CT), magnetic resonance imaging (MRI), x-ray, or ultrasound image data set. Alternatively, the surgical plan may be created using data collected directly from the patient intraoperatively (e.g. digitized points, kinematic femoral head center, ankle center, statistical bone morphing) such as with typical imageless navigation systems rather than using a pre-operative image data set. A set of 3-D computer aided design (CAD) models of the manufacturer's prosthesis (i.e. virtual implant models) are pre-loaded in the software that allows the user to place the components of a desired prosthesis to the 3-D model of the boney anatomy or the intraoperatively collected data to designate the best fit, position and orientation of the implant to the bone. For example, with reference to FIG. 3, a 3-D model of the patient's distal femur 180 and a 3-D model of the femoral prosthesis 182 are shown. The final placement of the prosthesis model 182 on the bone model 180 defines the bone cut planes (shaded regions of the bone model 180) where the bone is cut intra-operatively to receive the prosthesis as desired. In TKA, the planned cut planes generally include the anterior cut plane 184, anterior chamfer cut plane 186, the distal cut plane 188, the posterior chamfer cut plane 190, the posterior cut plane 192 and the tibial cut plane (not shown).

In a particular embodiment, the surgical plan contains the 3-D model of the patient's operative bone combined with the location of one or more virtual planes 194 as described in co-pending provisional application 62/259,487 assigned to the assignee of the present application and incorporated by reference herein in its entirety. The location of the virtual pin plane(s) 194 is defined by the planning software using the POSE of one or more planned cut planes and one or more dimensions of a cutting guide (not shown) used in conjunction with the surgical device 102.

It is also contemplated that embodiments of the present invention may further include any of the following: a surgical plan containing a cut file specifying instructions for a manipulator arm to autonomously create the planar cuts on the bone; a surgical plan containing a virtual boundary that provides haptic feedback or parameterization control to a user while wielding a surgical device (e.g., a manipulator arm, a hand-held device) to maintain the device within that boundary; conventional manual jigs or adjustable cutting guides which may or may not be tracked in the operating workspace; a surgical plan having a combination of autonomous and haptic instructions for a surgical system; and a surgical plan having instructions for a bone mounted robot to execute the procedure on the bone.

### Bone Motion Monitoring

The tracked fiducial marker array 18 on the knee brace 11 and the one or more single-LED markers 20 may also be used as a bone motion monitor. In a particular embodiment, certain motions of the one or more single-LED markers 20 on the bone relative to the fiducial marker array 18 on the brace 11 might indicate that the patient moved too much in the brace 11 to continue tracking accurately. Even if it is unknown how the bone moved in the brace, it may still be useful as a bone motion monitor to trigger an event. The event may be a simple notification to the user. The notification may instruct the user to re-register the bone or re-collect points on the bone. The event may be executed by the computing system 104 to control the surgical device such as freezing the robotic arm or removing power from the operating tool.

### Other Embodiments

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the described embodiments in any way. Rather, the foregoing detailed description will provide those skilled in the art with a convenient roadmap for implementing the exemplary embodiment or exemplary embodiments. It should be understood that various changes may be made in the function and arrangement of elements without departing from the scope as set forth in the appended claims and the legal equivalents thereof.

1. A system for tracking a bone of a subject, comprising:
a brace with a securement adapted for holding a portion of a limb of the subject;
a fiducial array with a series of fiducial markers attached to said brace;
one or more single-LED (light emitting diode) markers each individually suspended by an extension, where said extension is inserted into a portion of the bone of the subject;
a tracking system adapted to track position and orientation (POSE) of said fiducial array and said one or more single-LED markers; and
a computing system, interfaced with the tracking system, programmed to:
store at least one of a position and an orientation of one or more reference features with respect to said fiducial array on said brace; and
compensate for the remaining degrees of freedom between said brace and the one or more reference features based on the tracked spatial relationship between said fiducial array on said brace and the one or more single-LED markers inserted on the bone.

2. The system of claim 1 wherein said one or more single-LED markers are a plurality of single-LED markers corresponding to at least a number of remaining degrees of freedom between said brace and said one or more points.

3. The system of claim 1 wherein the one or more reference features is one or more points collected on the bone.

4. The system of claim 1 wherein the one or more reference features is a virtual bone model registered to the bone.

5. The system of claim 4 wherein at least one of a cut file, a bounding volume, or a haptic boundary are defined with respect to the virtual bone model.

6. The system of claim 1 wherein the one or more reference features is a virtual implant model.

7. The system of claim 6 wherein the one or more reference features is a virtual implant model planned relative to at least one a virtual bone model, one or more points collected on the bone, or a statistically shaped bone model.

8. The system of claim 1 wherein said extension is a tack or a screw.

9. The system of claim 1 wherein said extension has a diameter less than 3 mm.

10. A method for tracking a bone of a subject comprising:
placing a portion of a limb of the subject in said brace of claim 1, and securing the portion of the limb with said securement to establish a position and orientation of said fiducial marker array;
inserting said one or more single-LED (light emitting diode) markers with said extensions into the subject's bone;
activating said tracking system to track the position and orientation (POSE) of said marker array and said one or more single-LED;
storing at least one of a position and orientation of one or more reference features with respect to said marker array on said brace for use as a base reference frame; and
tracking the bone, where said one or more single-LED markers are used to compensate for the remaining degrees of freedom between said brace and the one or more reference features.

11. The method of claim 10 further comprising collecting one or more points on the bone, wherein the one or more points are stored as the one or more reference features.

12. The method of claim 10 further comprising collecting one or more points on the bone; planning a placement of a virtual implant model with respect to the one or more points; wherein the virtual implant model is stored as the one or more reference features.

13. The method of claim 10 wherein the bone one or more reference features is a virtual model of a bone registered to the bone.

14. A system for monitoring motion of a bone of a subject during a surgical procedure, comprising:
a brace with a securement adapted for holding a portion of a limb of the subject;
a fiducial array with a series of fiducial markers fixedly attached to said brace;
one or more single-LED (light emitting diode) markers each individually suspended by an extension, where said extension is inserted into a portion of the bone;
a tracking system adapted to track position and orientation (POSE) of said fiducial array and said one or more single-LED markers; and
a computing system, interfaced with the tracking system, programmed to:
store one or more reference features with respect to said fiducial array on said brace; and
notify a user if the tracking system detects motion between the one or more single-LED markers and said fiducial array.
